(19) United States
(12) Patent Application Publication
(10) Pub. No.: US 2007/0258976 A1
(43) Pub. Date: Nov. 8, 2007

(54) Combination Therapy for Diseases Involving Angiogenesis

(76) Inventors: Keith W. Ward, Ontario, NY (US); Praveen Tyle, Pittsford, NY (US)

Correspondence Address: Bausch & Lomb Incorporated, One Bausch & Lomb Place, Rochester, NY, 14604-2701, US

(21) Appl. No.: 11/733,282

(22) Filed: Apr. 10, 2007

Related U.S. Application Data
(60) Provisional application No. 60/797,608, filed on May 4, 2006.

(51) Int. Cl. A61K 39/395

(52) U.S. Cl. 424/130.1

(57) ABSTRACT

A composition useful for treating, preventing, or ameliorating a disease condition involving abnormal angiogenesis comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF. The invention also includes a method for treating, preventing, or ameliorating a disease condition involving abnormal angiogenesis using such a composition.

### CROSS-REFERENCE

This application claims the benefit of Provisional Patent Application No. 60/797,608 filed May 4, 2006, which is incorporated by reference herein.

### BACKGROUND OF THE INVENTION

The present invention relates to compositions and methods for preventing, treating, or ameliorating conditions of diseases involving angiogenesis. In particular, the present invention relates to such compositions and methods that target two or more modes of action of vascular endothelial growth factor (“VEGF”) in such diseases. More particularly, the present invention relates to such compositions and methods that target two or more modes of action of VEGF in ocular diseases involving angiogenesis.

Every year, more than six million patients are newly diagnosed with serious ocular illnesses. Neovascularization in the eye is associated with various ocular disorders, often causing severe loss of vision and eventually blindness. Among these disorders, diabetic retinopathy (“DR”) and age-related macular degeneration (“AMD”) are the most prevalent. DR is seen in the 16 million American patients with diabetes, one third of whom remain undiagnosed and untreated. AMD is the leading cause of visual loss in people 65 years and older. As the population ages, the incidence of AMD will most likely increase.

DR affects the inner retina while AMD affects the outer retina and retinal pigment in the epithelium. In DR, the blood supply to the inner part of the retina is impaired. The eye's blood vessels leak and close off. Cells in the eye then signal for new vessel growth by releasing angiogenic factors. As new vessels grow in response to these factors, they bleed and contract as well, causing scar tissues that can eventually lead to detachment of the retina and blindness.

AMD appears as a sudden worsening and distortion of the central vision that progresses rapidly. This disease typically has a preclinical, asymptomatic phase, in which extracellular waste material accumulates in the space between the Bruch's membrane and the epithelial layer, forming yellow-white spots known as drusen. Advanced forms of AMD include both dry and wet (or exudative) AMD. The dry form of AMD is far more common, and the wet form occurs simultaneously with the dry form in about 15% of the cases. Dry AMD is characterized by progressive apoptosis of cells in the epithelial layer, in the overlying photoreceptor layer, and in the underlying cells in the choroidal capillary layer because of deprivation of nourishment due to insufficient circulation. As a defensive mechanism, surviving cells release angiogenic factors to stimulate growth of new vessels from the choroidal vessels. These new vessels are typically leaky, and as a result, fluid accumulates in the subretinal space, leading to separation of the retina from the underlying layers.

Several endogenous proteins have been implicated in the regulation of angiogenesis. Among these angiogenic factors are acidic fibroblast growth factor (“aFGF”), basic fibroblast growth factors (“bFGF”), transforming growth factor-α (“TGF-α”), transforming growth factor-β (“TGF-β”), hepatocyte growth factor (“HGF”), tumor necrosis factor-α (“TNF-α”), platelet derived growth factor (“PDGF”), angiogenin, interleukin-8 (“IL-8”), etc. Although these molecules have been shown to promote angiogenesis, at least in certain model systems, it has been difficult consistently to correlate their activity with the physiological or pathological regulation of blood vessel growth. (N. Ferrara and T. Davis-Smyth, Endocrine Rev., Vol.18, No.1, 4 (1997). On the other hand, evidence accumulating over the past decade has strongly suggested that vascular endothelial growth factor (“VEGF”) is a key promoter of abnormal ocular angiogenesis (See; e.g., L. P. Aiello et al., New Engl. J. Med., Vol. 331, No. 22, 1480 (1994); J. W. Miller et al., Am. J. Pathol., Vol.145, No. 3, 574 (1994); L. P. Aiello et al., Proc. Natl. Acad. Sci., Vol. 92,10457 (1995); A. N. Witmer et al., Prog. Ret. and Eye Res., Vol. 22,1 (2003)).

Considerable evidence has pointed to hypoxia as an important stimulus for VEGF production in both malignant and normal cells through an increase in the rate of VEGF gene transcription and an enhancement of the stability of VEGF mRNA (G. M. McMahon, The Oncologist, Vol. 5, Suppl. 1, 3 (2000)). An increased level of VEGF leads to an increased binding of this molecule to the VEGF receptor tyrosine kinases (“RTKs”) on endothelial cell surface, activating and expanding the catalytic cascades of signal transduction pathways that promote cell proliferation, differentiation, migration, and metabolism, including those of endothelial cells. Thus, an increased level of VEGF production can be the beginning of aberrant angiogenesis.

Efforts have been devoted to control abnormal angiogenesis by controlling the level of VEGF or blocking or inhibiting the action of VEGF. For example, Macugen® (pegaptanib sodium injection, developed by EyeTech), the active ingredient of which is a PEGylated aptamer that binds to and inhibits the function of VEGF has been approved by the US FDA for the treatment of AMD. Lucentis™ (ranibizumab, developed by Genetech), a recombinant antibody against VEGF, has been the subject of two clinical trials with some success. Another approach has been to provide VEGF-receptor inhibitors, which bind to the VEGF receptors and render them unavailable for activation by VEGF. Several of these inhibitors (such as SU5416 by Sugen, ZD4190 by AstraZeneca, and ZK222584 and CGP41251 by Novartis) are being tested for treatment of tumor angiogenesis (G. M. McMahon, supra). However, a therapy relying on supplying a compound to target one single event in the VEGF-induced angiogenic cascade would require a large dose of the compound to be effective. Such large doses may be toxic to the patients. In addition, targeting only a single event in the VEGF-induced angiogenic cascade may not completely disrupt the cascade, thus such therapy may not be completely effective.

Therefore, there is a continued need to provide improved compositions and methods for preventing, treating, or ameliorating conditions of diseases involving angiogenesis. In addition, it is very desirable to provide such compositions and methods that can be effective and safe to patients.

### SUMMARY OF THE INVENTION

In general, the present invention provides compositions and methods for preventing, treating, or ameliorating conditions of diseases involving angiogenesis.

In one aspect, the present invention provides such compositions and methods that target two or more sources of VEGF activity in such diseases, leading at least to a reduction in an availability of active VEGF.

In another aspect, the present invention provides such compositions and methods that target two or more sources of VEGF activity in ocular diseases.

In still another aspect, a composition of the present invention comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

In yet another aspect, compounds that interact with and inhibit a downstream activity of extracellular VEGF are capable of binding to extracellular VEGF and rendering it incapable of participating in a VEGF-induced angiogenic cascade.

In still another aspect of the present invention, compounds that interact with at least a VEGF receptor are capable of competitively binding to said at least a VEGF receptor and rendering such VEGF receptor substantially incapable of binding with VEGF. Such compounds, in one aspect, block, inhibit, modulate, or regulate a VEGF-dependent tyrosine kinase signal transduction.

In a further aspect of the present invention, compounds that reduce a level of expression of VEGF are capable of interfering with at least a step in the chain of events leading to the expression of VEGF.

In still another aspect, a composition of the present invention is used for preventing, treating, or ameliorating a disease condition involving angiogenesis. In one embodiment, such a disease condition involves abnormal ocular angiogenesis.

In yet another aspect, the present invention provides a method for preventing, treating, or ameliorating a disease condition involving angiogenesis. The method comprises administering to a subject in need of preventing, treating, or ameliorating the disease condition a therapeutically effective amount of a composition that comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

In a further aspect, such a disease condition is selected from the group consisting of diabetic edema (“DE”), DR, AMD, and combinations thereof.

Other features and advantages of the present invention will become apparent from the following detailed description and claims and the appended drawings.

### DETAILED DESCRIPTION OF THE INVENTION

In general, the present invention provides compositions and methods for preventing, treating or ameliorating conditions of diseases involving angiogenesis.

In one aspect, the present invention provides such compositions and methods that target two or more sources of VEGF activity in such diseases, leading at least to a reduction in an availability of active VEGF.

In another aspect, the present invention provides such compositions and methods that target two or more sources of VEGF activity in ocular diseases. In one embodiment, such compositions or methods can substantially eliminate, reduce, or inhibit the production or availability of active VEGF.

In still another aspect, the present invention provides such compositions and methods that target two or more sources of VEGF activity in disease conditions that support tumor growth, such as those exhibiting rapid and wide-spread abnormal angiogenesis.

Accumulating evidence has established that excessive activity of VEGF plays a key role in pathological angiogenesis. VEGF is a secreted disulfide-linked homodimer that selectively stimulates endothelial cells to proliferate, migrate, and produce matrix-degrading enzymes, all of which are required for the formation of new blood vessels. In addition to being the only known endothelial cell specific mitogen, VEGF is unique among angiogenic growth factors in its ability to induce a transient increase in blood vessel permeability to macromolecules (hence its original and alternative name, vascular permeability factor or “VPF”). Increased vascular permeability and the resulting deposition of plasma proteins in the extravascular space assist the new vessel formation by providing a provisional matrix for the migration of endothelial cells. Hyperpermeability is a characteristic feature of new vessels, including those associated with tumors. Furthermore, compensatory angiogenesis induced by tissue hypoxia is now known to be mediated by VEGF.

VEGF exists in four forms (VEGF₁₂₁, VEGF₁₆₅, VEGF₁₈₉, and VEGF₂₀₆) as a result of alternative splicing of the VEGF gene. The two smaller forms are diffusible while the larger two forms remain predominantly localized to the cell membrane as a consequence of their high affinity for heparin. VEGF₁₆₅ also binds to heparin and is the most abundant form. VEGF₁₂₁, the only form that does not bind to heparin, appears to have a lower affinity for the VEGF receptors as well as lower mitogenic potency. The biological effects of VEGF are mediated by two tyrosine kinase receptors: Flt-1 (or VEGFR-1) and Flk-1/KDR (or VEGFR-2), the expression of which is highly restricted to cells of endothelial origin (N. Ferrara, Am. J. Physiol. Cell Physiol, Vol. 280, C1358 (2001)). While the expression of both functional receptors is required for high affinity binding, the chemotactic and mitogenic signaling in endothelial cells appears to occur primarily through the KDR (or VEGFR-2) receptor. The importance of VEGF and VEGF receptors for the development of blood vessels has recently been demonstrated in mice lacking a single allele for the VEGF gene (Carmeliet et al., Nature, Vol. 380, 435 (1996); Ferrara et al., Nature, Vol. 380, 439 (1996)) or both alleles of the Flt-1 gene (Fong et al., Nature, Vol. 376, 66 (1995)) or Flk-1/KDR gene (Shalaby et al., Nature, Vol. 376, 62 (1995)). In each case, distinct abnormalities in vessel formation were observed resulting in embryonic lethality.

VEGFR-1 and VEGFR-2 belong to the class of transmembrane receptor-type tyrosine kinases, which catalyze the transfer of the terminal phosphate of adenosine triphosphate to tyrosine residues in protein substrates. Many of these proteins act as enzymes in cellular physiological processes. Thus, tyrosine kinases in general, and VEGFR-1 and VEGFR-2 in particular, play critical roles in signal transduction for a number of cell functions, such as cell proliferation, differentiation, migration, etc. Therefore, elevated levels of VEGF and VEGF receptors that are free to interact with each other are key contributing factors for abnormal angiogenesis.

In one aspect, a composition of the present invention comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

### 1. Compounds that Interact with and Inhibit a Downstream Activity of Extracellular VEGF

In one aspect of the present invention, compounds that interact with and inhibit a downstream activity of extracellular VEGF are capable of binding to extracellular VEGF and rendering it incapable of participating in a VEGF-induced angiogenic cascade.

In one embodiment, compounds that interact with and inhibit a downstream activity of extracellular VEGF comprise a nucleic acid ligand that binds to extracellular VEGF and substantially prevents it from participating in the angiogenic cascade. Non-limiting examples of such a nucleic acid ligand are the VEGF aptamers disclosed in U.S. Pat. Nos. 6,426,335; 6,168,778; 6,147,204; 6,051,698; and 6,011,020; which are incorporated herein by reference in their entirety. In one embodiment, such a nucleic acid ligand comprises the VEGF antagonist aptamer known by its trade name “Macugen®”, being marketed by OSI EyeTech Pharmaceuticals (Melleville, N.Y.). The aptamer binds and inactivates VEGF in a manner similar to that of a high-affinity antibody directed toward VEGF. In one embodiment, the aptamer binding renders VEGF incapable of binding to VEGF receptors. In another embodiment, the VEGF receptors comprise those bound to or expressed at cell surface, such as VEGFR-1 and/or VEGFR-2.

In another aspect, compounds that bind to extracellular VEGF and render it incapable of participating in a VEGF-induced angiogenic cascade comprise an anti-VEGF antibody or antibody fragment. Non-limiting examples of anti-VEGF antibodies include those disclosed in U.S. Pat. Nos. 5,730,977; 6,100,071; 6,342,221; and 6,582,959; the contents of which are incorporated herein by reference in their entirety. In one embodiment, anti-VEGF antibodies include those directed against VEGF or its cognate receptors (VEGFR-1, VEGFR-2, or both).

Anti-VEGF antibodies useful in the present invention include monoclonal inhibitory antibodies. Monoclonal antibodies, or fragments thereof, encompass all immunoglobulin classes such as IgM, IgG, IgD, IgE, IgA, or their subclasses, such as the IgG subclasses or mixtures thereof. Fragments of antibodies that are useful are truncated or modified antibody fragments with one or two antigen-complementary binding sites that show high binding and neutralizing activity toward mammalian VEGF (or its cognate receptors), such as parts of antibodies having a binding site and is formed by light and heavy chains, such as Fv, Fab or F(ab)₂ fragments, or single-stranded fragments. In some embodiments, truncated double-stranded fragments such as Fv, Fab or F(ab)₂ are useful. These fragments can be obtained, for example, by enzymatic means by eliminating the Fc part of the antibody with enzymes such as papain or pepsin, by chemical oxidation or by genetic manipulation of the antibody genes. It is also possible and advantageous to use genetically manipulated, non-truncated fragments. The anti-VEGF antibodies or fragments thereof can be used alone or in mixtures.

The anti-VEGF antibodies, antibody fragments, mixtures, or derivatives thereof advantageously have a binding affinity for VEGF (or its cognate receptors) in a range from 1×10⁻⁷ M to 1×10⁻¹² M, or from 1×10⁻⁸ M to 1×10⁻¹¹ M, or from 1×10⁻⁹ M to 5×10⁻¹⁰ M.

The present invention further includes derivatives of anti-VEGF antibodies, which substantially retain their VEGF-inhibiting activity while altering one or more other properties related to their use as a pharmaceutical agent; e.g., serum stability or efficiency of production. Non-limiting examples of such anti-VEGF antibody derivatives include peptides, peptidomimetics derived from the antigen-binding regions of the antibodies, and antibodies, antibody fragments or peptides conjugated to another physiologically acceptable material, such as polyethylene glycol, synthetic polymers such as polyacrylamide, polyacrylic acid, polymethacrylic acid, or natural polymers or derivatives thereof such as cellulose, Sepharose™ or agarose, or conjugates with enzymes.

The anti-VEGF monoclonal antibodies of the present invention may be obtained by any means known in the art. For example, in one embodiment, a mammal is immunized with human VEGF (or their cognate receptors) anti-VEGF antibodies are obtained therefrom. In another embodiment, such antibodies are further “humanized,” as disclosed below. Purified human VEGF is commercially available (e.g., from Cell Sciences, Norwood, Mass.). Alternatively, human VEGF (or their cognate receptors) may be readily purified from human placental tissue.

The monoclonal antibodies can include hybrid and recombinant antibodies produced by splicing a variable (including hypervariable) domain of an anti-VEGF antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. See; e.g., U.S. Pat. No. 4,816,567 for a method of making fusion protein, which Patent is incorporated herein by reference in its entirety.

In cases in which such antibodies or antibody fragments are obtained from non-human sources, it is desirable to provide “humanized” forms of such antibodies or antibody fragments in a composition of the present invention. “Humanized” forms of non-human (e.g., murine) antibodies are specific chimeric immunoglobulins, immunoglobulin chains, or fragments thereof (such as Fv, Fab, Fab′, F(ab)₂ or other antigen-binding subsequences of antibodies) that contain minimal sequence derived from non-human immunoglobulin. For the most part, humanized antibodies are human immunoglobulins (recipient antibody) in which residues from the complementary determining regions (“CDRs”) of the recipient antibody are replaced by residues from the CDRs of a non-human species (donor antibody) such as mouse, rat, or rabbit having the desired specificity, affinity and capacity. Methods for humanizing non-human antibodies are well known in the art. Generally, a humanized antibody has a sequence of amino acid residues introduced into it from a non-human source. See; e.g., Jones et al., Nature, Vol. 321, 522 (1986); Riechmann et al., Nature, Vol. 332, 323 (1988); and Verhoeyen et al., Science, Vol. 239,1534 (1988).

In one embodiment, an anti-VEGF antibody of the composition can be the recombinant monoclonal antibody known as Lucentis™ (ranibizumab, developed by Genentech, South San Francisco, Calif.).

### 2. Compounds that Interact with at Least a VEGF Receptor and Render it Substantially Unavailable for Interacting with VEGF

In one aspect of the present invention, compounds that interact with at least a VEGF receptor and render it substantially unavailable for interacting with VEGF comprises VEGF tyrosine kinase inhibitors, which can be a small synthetic molecule or protein or protein fragment that binds to the transmembrane VEGF receptors and neutralizes their activation, such as rendering them incapable of initiating or participating further in the expression of VEGF or other angiogenic factors.

Non-limiting examples of synthetic VEGF tyrosine kinase inhibitors include the compounds disclosed in U.S. Pat. No. 6,958,340, which is incorporated herein by reference in its entirety. These compounds are characterized in that they comprise pyrimidine or substituted pyrimidine linked to imidazole or substituted imidazole. Non-limiting examples of this type of tyrosine kinase inhibitors include 4-(2-phenyl-1H-imidazol-1-yl)-N-pyridin-4-ylpyrimidin-2-amine; 4-(2-phenyl-1H-imidazol-1-yl)-N-pyrimidin-4-ylpyrimidin-2-amine; 4-(2-phenyl-1H-imidazol-1-yl)-N-pyrimidin-2-ylpyrimidin-2-amine; 4-(2-phenyl-1H-imidazol-1-yl)-N-pyrazin-2-ylpyrimidin-2-amine; 4-(2-phenyl-1H-imidazol-1-yl)-N-(1,3,4-thiadiazol-2-yl)pyrimidin-2-amine; N-(5-methyl-1,3,4-thiadiazol-2-yl)-4-(2-phenyl-1H-imidazol-1-yl)pyrimidin-2-amine; N-isoxazol-3-yl-4-(2-phenyl-1H-imidazol-1-yl)pyrimidin-2-amine; N-(3-methylisoxazol-5-yl)-4-(2-phenyl-1H-imidazol-1-yl)pyrimidin-2-amine; N-(4-methyl-1,3-thiazol-2-yl)-4-(2-phenyl-1H-imidazol-1-yl)pyrimidin-2-amine; N-(2-methylpyridin-4-yl)-4-(2-phenyl-1H-imidazol-1-yl)pyrimidin-2-amine; N-(2,6-dimethylpyridin-4-yl)-4-(2-phenyl-1H-imidazol-1-yl)pyrimidin-2-amine; 4-(2-phenyl-1H-imidazol-1-yl)-N-pyridin-3-ylpyrimidin-2-amine; N-(1-oxidopyndin-3-yl)-4-(2-phenyl-1H-imidazol-1-yl)pyrimidin-2-amine; N-{3-methoxy-5-(trifluoromethyl)phenyl}-4-(2-phenyl-1H-imidazol-1-yl)pyrimidin-2-amine; (3-methyl-5-{[4-(2-phenyl-1H-imidazol-1-yl)pyrimidin-2-yl]amino}phenyl)methanol; N-{3-[(4-acetylpiperazin-1-yl)methyl]-5-methylphenyl}-4-(2-phenyl-1H-imidazol-1-yl)pyrimidin-2-amine; N-(3,5-dimethylphenyl)-4-(2-pyridin-2-yl-1H-imidazol-1-yl)pyrimidin-2-amine; N-(3,5-dimethylphenyl)-4-(2-pyrimidin-5-yl-1H-imidazol-1-yl)pyrimidin-2-amine; N-(3,5-dimethylphenyl)-4-(2-pyridin-3-yl-1H-imidazol-1-yl)pyrimidin-2-amine; 4-(2-cyclopropyl-1H-imidazol-1-yl)-N-(3,5-dimethylphenyl)pyrimidin-2-amine; N-(3,5-dimethylphenyl)-4-(4-methyl-2-phenyl-1H-imidazol-1-yl)pyrimidin-2-amine; 1-{2-[(3,5-dimethylphenyl)amino]pyrimidin-4-yl}-1H-imidazole-2-carbonitrile; N-(3,5-dimethylphenyl)-4-(2-methyl-1H-imidazol-1-yl)pyrimidin-2-amine; 4-(2-amino-1H-imidazol-1-yl)-N-(3,5-dimethylphenyl)pyrimidin-2-amine; N-(2-methylphenyl)-4-(2-phenyl-1H-imidazol-1-yl)pyrimidin-2-amine; N-(2-methoxyphenyl)-4-(2-phenyl-1H-imidazol-1-yl)pyrimidin-2-amine; N-(2-fluorophenyl)-4-(2-phenyl-1H-imidazol-1-yl)pyrimidin-2-amine; N-(3-chlorophenyl)-4-(2-phenyl-1H-imidazol-1-yl)pyrimidin-2-amine; N-(3,5-dichlorophenyl)-4-(2-phenyl-1H-imidazol-1-yl)pyrimidin-2-amine; N-(3-fluorophenyl)-4-(2-phenyl-1H-imidazol-1-yl)pyrimidin-2-amine; N-(3-methoxyphenyl)-4-(2-phenyl-1H-imidazol-1-yl)pyrimidin-2-amine; N-(3-methylphenyl)-4-(2-phenyl-1H-imidazol-1-yl)pyrimidin-2-amine; N-(3,5-dimethoxyphenyl)-4-(2-phenyl-1H-imidazol-1-yl)pyrimidin-2-amine; N-(4-chlorophenyl)-4-(2-phenyl-1H-imidazol-1-yl)pyrimidin-2-amine; N-(4-fluorophenyl)-4-(2-phenyl-1H-imidazol-1-yl)pyrimidin-2-amine; N-(4-methoxyphenyl)-4-(2-phenyl-1H-imidazol-1-yl)pyrimidin-2-amine; N-(4-methylphenyl)-4-(2-phenyl-1H-imidazol-1-yl)pyrimidin-2-amine; N-[3,5-bis(trifluoromethyl)phenyl]-4-(2-phenyl-1H-imidazol-1-yl)pyrimidin-2-amine; N-[3-methyl-5-(trifluoromethyl)phenyl]-4-(2-phenyl-1H-imidazol-1-yl)pyrimidin-2-mine; N-(3,5-difluorophenyl)-4-(2-phenyl-1H-imidazol-1-yl)pyrimidin-2-amine; 4-(2-phenyl-1H-imidazol-1-yl)-N-[3-(trifluoromethyl)phenyl]pyrimidin-2-amine; N-(3,5-dimethylphenyl)-4-(2-phenyl-1H-imidazol-1-yl)pyrimidin-2-amine; 4-(2-phenyl-1H-imidazol-1-yl)-N-pyridin-2-ylpyrimidin-2-amine; N-{3-[(4-ethylpiperazin-1-yl)methyl]phenyl)-4-(2-phenyl-1H-imidazol-1-yl)pyrimidin-2-amine; 4-(2-chloro-1H-imidazol-1-yl)-N-(3,5-dimethylphenyl)pyrimidin-2-amine; and N-(3,5-dimethylphenyl)4-[2-(3-fluorophenyl)-1H-imidazol-1-yl]pyrimidin-2-amine.

Other non-limiting examples of synthetic VEGF tyrosine kinase inhibitors include the quinazoline derivatives disclosed in U.S. Patent Application Publication 2005/0245549, which is incorporated herein by reference in its entirety. For example, two such quinazoline derivatives are shown below.

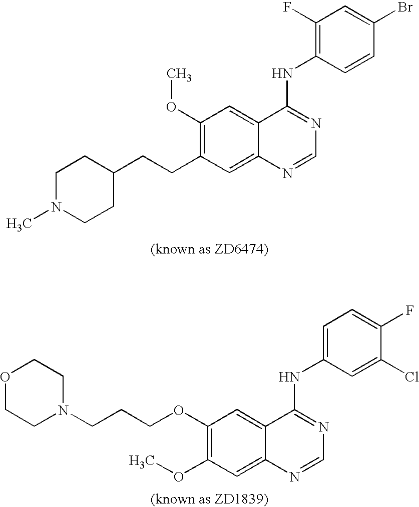

Other non-limiting examples of tyrosine kinase inhibitors of the type of quinazoline derivatives are disclosed in U.S. Patent Application Publication 2006/0058523 and include (1-(3-((4-((5-(2-((3-fluorophenyl)amino)-2-oxoethyl)-1,3-thiazol-2-yl)amino)-6-methoxyquinazolin-7-yl)oxy)propyl)piperidin-4-yl)methyl dihydrogen phosphate; ((2R)-1-(3-((4-((5-(2-((3-fluorophenyl)amino)-2-oxoethyl)-1,3-thiazol-2-y-l)amino)-6-methoxyquinazolin-7-yl)oxy)propyl)pyrrolidin-2-yl)methyl dihydrogen phosphate; 2-(4-(3-((4-((5-(2-((3-fluorophenyl)amino)-2-oxoethyl)-1,3-thiazol-2-yl)amino)-6-methoxy-quinazolin-7-yl)oxy)propyl)piperazin-1-yl)ethyl dihydrogen phosphate; 1-(3-((4-((5-(2-((3-fluorophenyl)amino)-2-oxoethyl)-1,3-thiazol-2-yl)amino)-6-methoxyquinazolin-7-yl)oxy)propyl)pyrrolidin-3-yl dihydrogen phosphate; 1-(3-((4-((5-(2-((3-fluorophenyl)amino)-2-oxoethyl)-1,3-thiazol-2-yl)amino)-6-methoxyquinazolin-7-yl)oxy)propyl)piperidin-3-yl dihydrogen phosphate; 2-(ethyl(3-((4-((5-(2-((3-fluorophenyl)amino)-2-oxoethyl)-1,3-thiazol-2-y-l)amino)-6-methoxyquinazolin-7-yl)oxy)propyl)amino)ethyl dihydrogen phosphate; ((2S)-1-(3-((4-((5-(2-((3-fluorophenyl)amino)-2-oxoethyl)-1,3-thiazol-2-y-l)amino)-6-methoxyquinazolin-7-yl)oxy)propyl)pyrrolidin-2-yl)methyl dihydrogen phosphate; 2-(ethyl(((2S)-1-(3-((4-((5-(2-((3-fluorophenyl)amino)-2-oxoethyl)-1,3-th-iazol-2-yl)amino)-6-methoxyquinazolin-7-yl)oxy)propyl)pyrrolidin-2-yl)methyl)amino)ethyl dihydrogen phosphate; 1-(3-((4-((5-(2-((3-fluorophenyl)amino)-2-oxoethyl)-1,3-thiazol-2-yl)amino)-6-methoxyquinazolin-7-yl)oxy)propyl)piperidin-4-yl dihydrogen phosphate; 2-((((2S)-1-(3-((4-((5-(2-((3-fluorophenyl)amino)-2-oxoethyl)-1,3-thiazol--2-yl)amino)-6-methoxyquinazolin-7-yl)oxy)propyl)pyrrolidin-2-yl)methyl)amino)ethyl dihydrogen phosphate; 2-((3-((4-((5-(2-((3-fluorophenyl)amino)-2-oxoethyl)-1,3-thiazol-2-yl)amino)-6-methoxyquinazolin-7-yl)oxy)propyl)amino)ethyl dihydrogen phosphate; 3-(ethyl(3-((4-((5-(2-((3-fluorophenyl)amino)-2-oxoethyl)-1,3-thiazol-2-y-l)amino)-6-methoxyquinazolin-7-yl)oxy)propyl)amino)propyl dihydrogen phosphate; 2-((2-fluoroethyl)(3-((4-((5-(2-((3-fluorophenyl)amino)-2-oxoethyl)-1,3-thiazol-2-yl)amino)-6-methoxyquinazolin-7-yl)oxy)propyl)amino)ethyl dihydrogen phosphate; 2-(1-(3-((4-((5-(2-((3-fluorophenyl)amino)-2-oxoethyl)-1,3-thiazol-2-yl)amino)-6-methoxyquinazolin-7-yl)oxy)propyl)piperidin-4-yl)ethyl dihydrogen phosphate; 2-((3-((4-((5-(2-((3-fluorophenyl)amino)-2-oxoethyl)-1,3-thiazol-2-yl)amino)-6-methoxyquinazolin-7-yl)oxy)propyl)(2-methoxyethyl)amino)ethyl dihydrogen phosphate; 2-((2S)-1-(3-((4-((5-(2-((3-fluorophenyl)amino)-2-oxoethyl)-1,3-thiazol-2-yl)amino)-6-methoxyquinazolin-7-yl)oxy)propyl)pyrrolidin-2-yl)ethyl dihydrogen phosphate; 2-((3-((4-((5-(2-((3-fluorophenyl)amino)-2-oxoethyl)-1,3-thiazol-2-yl)amino)-6-methoxyquinazolin-7-yl)oxy)propyl)amino)-2-methylpropyl dihydrogen phosphate; ((2R)-1-(3-((4-((5-(2-((3-chlorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; 2-(1-(3-((4-((5-(2-((3-chlorophenyl)amino)-2-oxoethyl)-1,3-thiazol-2-yl)amino)-6-methoxyquinazolin-7-yl)oxy)propyl)piperidin-4-yl)ethyl dihydrogen phosphate; 2-(4-(3-((4-((5-(2-(3,5-difluorophenyl)amino)-2-oxoethyl)-1,3-thiazol-2-yl)amino)-6-methoxyquinazolin-7-yl)oxy)propyl)piperazin-1-yl)ethyl dihydrogen phosphate; 2-((3-((4-((5-(2-((3,5-difluorophenyl)amino)-2-oxoethyl)-1,3-thiazol-2-yl)amino)-6-methoxyquinazolin-7-yl)oxy)propyl)(methyl)amino)ethyl dihydrogen phosphate; ((2S)-1-(3-((4-((5-(2-((3,5-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; (1R)-2-((3-((4-((5-(2-((3,5-difluorophenyl)amino)-2-oxoethyl)-1,3-thiazol--2-yl)amino)-6-methoxyquinazolin-7-yl)oxy)propyl)amino)-1-methylethyl dihydrogen phosphate; ((2R)-1-(3-((4-((5-(2-((3,4-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; ((2S)-1-(3-((4-((5-(2-((3,4-difluorophenyl)amino)-2-oxoethyl)-1,3-thiazol--2-yl)amino)-6-methoxyquinazolin-7-yl)oxy)propyl)pyrrolidin-2-yl)methyl dihydrogen phosphate; 1-(3-((4-((5-(2-((3,4-difluorophenyl)amino)-2-oxoethyl)-1,3-thiazol-2-yl)amino)-6-methoxyquinazolin-7-yl)oxy)propyl)piperidin-4-ylmethyl dihydrogen phosphate; (1-(3-((4-((5-(2-((2-fluorophenyl)amino)-2-oxoethyl)-1,3-thiazol-2yl)amino)-6-methoxyquinazolin-7-yl)oxy)propyl)piperidin-4-yl)methyl dihydrogen phosphate; ((2R)-1-(3-((4-((5-(2-((2-fluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; ((2S)-1-(3-((4-((5-(2-((2-fluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; 2-(ethyl(3-((4-((5-(2-((2-fluorophenyl)amino)-2-oxoethyl)-1,3-thiazol-2-yl)amino)-6-methoxyquinazolin-7-yl)oxy)propyl)amino)ethyl dihydrogen phosphate; 2-(1-(3-((4-((5-(2-((2-fluorophenyl)amino)-2-oxoethyl)-1,3-thiazol-2-yl)amino)-6-methoxyquinazolin-7-yl)oxy)propyl)piperidin-2-yl)ethyl dihydrogen phosphate; ((2R)-1-(3-((4-((5-(2-((2,3-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; ((2S)-1-(3-((4-((5-(2-((2,3-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; 2-((3-((4-((5-(2-((2,3-difluorophenyl)amino)-2-oxoethyl)-1,3-thiazol-2-yl)amino)-6-methoxyquinazolin-7-yl)oxy)propyl)(methyl)amino)ethyl dihydrogen phosphate; 2-(1-(3-((4-((5-(2-((2,3-difluorophenyl)amino)-2-oxoethyl)-1,3-thiazol-2-yl)amino)-6-methoxyquinazolin-7-yl)oxy)propyl)piperidin-2-yl)ethyl dihydrogen phosphate; 2-((3-((4-((5-(2-((2,3-difluorophenyl)amino)-2-oxoethyl)-1,3-thiazol-2-yl-)amino)-6-methoxyquinazolin-7-yl)oxy)propyl)(ethyl)amino)ethyl dihydrogen phosphate; 1-(3-((4-((5-(2-((2,3-difluorophenyl)amino)-2-oxoethyl)-1,3-thiazol-2-yl)amino)-6-methoxyquinazolin-7-yl)oxy)propyl)piperidin-4-ylmethyl dihydrogen phosphate; 2-(4-(3-((4-((5-(2-((2,3-difluorophenyl)amino)-2-oxoethyl)-1,3-thiazol-2-yl)amino)-6-methoxy-quinazolin-7-yl)oxy)propyl)piperazin-1-yl)ethyl dihydrogen phosphate; 3-((3-((4-((5-(2-((2,3-difluorophenyl)amino)-2-oxoethyl)-1,3-thiazol-2-yl)amino)-6-methoxyquinazolin-7-yl)oxy)propyl)amino)-3-methylbutyl dihydrogen phosphate; 2-((3-((4-((5-(2-((2,3-difluorophenyl)amino)-2-oxoethyl)-1,3-thiazol-2-yl)amino)-6-methoxyquinazolin-7-yl)oxy)propyl)amino)-2-methylpropyl dihydrogen phosphate; 2-((3-((4-((5-(2-((2,3-difluorophenyl)amino)-2-oxoethyl)-1,3-thiazol-2-yl)amino)-6-methoxyquinazolin-7-yl)oxy)propyl)amino)ethyl dihydrogen phosphate; ((2R)-1-(3-((4-((5-(2-((2,5-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; ((2S)-1-(3-((4-((5-(2-((2,5-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; 2-((3-((4-((5-(2-((2,5-difluorophenyl)amino)-2-oxoethyl)-1,3-thiazol-2-yl)amino)-6-methoxyquinazolin-7-yl)oxy)propyl)(ethyl)amino)ethyl dihydrogen phosphate; ((2S)-1-(3-((4-((5-(2-((2,4-difluorophenyl)amino)-2-oxoethyl)-1,3-thiazol-2-yl)amino)-6-methoxyquinazolin-7-yl)oxy)propyl)pyrrolidin-2-yl)methyl dihydrogen phosphate; 2-(1-(3-((4-((5-(2-((2,4-difluorophenyl)amino)-2-oxoethyl)-1,3-thiazol-2-yl)amino)-6-methoxyquinazolin-7-yl)oxy)propyl)piperidin-2-yl)ethyl dihydrogen phosphate; 2-{cyclopropyl[3-({4-[(5-{2-[(3-fluorophenyl)amino]-2-oxoethyl}-1,3-thiazol-2-yl)amino]-6-methoxyquinazolin-7-yl}oxy)propyl]amino}ethyl dihydrogen phosphate; 2-{cyclopropyl[3-({4-[(5-{2-[(2,3-difluorophenyl)amino]-2-oxoethyl}-1,3-thiazol-2-yl)amino]-6-methoxyquinazolin-7-yl}oxy)propyl]aminoethyl dihydrogen phosphate; (1-(2-((4-((5-(2-((2,3-difluorophenyl)amino)-2-oxoethyl)-1,3-thiazol-2-yl)amino)-6-methoxyquinazolin-7-yl)oxy)ethyl)piperidin-4-yl)methyl dihydrogen phosphate; ((2R)-1-(2-((4-((5-(2-((2,3-difluorophenyl)amino)-2-oxoethyl)-1,3-thiazol-2-yl)amino)-6-methoxyquinazolin-7-yl)oxy)ethyl)pyrrolidin-2-yl)methyl dihydrogen phosphate; 2-(4-(2-((4-((5-(2-(2,3-difluorophenyl)amino)-2-oxoethyl)-1,3-thiazol-2-yl)amino)-6-methoxyquinazolin-7-yl)oxy)ethyl)piperazin-1-yl)ethyl dihydrogen phosphate; 2-(1-(2-((4-((5-(2-((3-fluorophenyl)amino)-2-oxoethyl)-1,3-thiazol-2-yl)amino)-6-methoxyquinazolin-7-yl)oxy)ethyl)piperidin-2-yl)ethyl dihydrogen phosphate; 2-(1-(2-((4-((5-(2-((3-fluorophenyl)amino)-2-oxoethyl)-1,3-thiazol-2-yl)amino)-6-methoxyquinazolin-7-yl)oxy)ethyl)piperidin-4-yl)ethyl dihydrogen phosphate; 4-(ethyl(2-((4-((5-(2-((3-fluorophenyl)amino)-2-oxoethyl)-1,3-thiazol-2-yl)amino)-6-methoxyquinazolin-7-yl)oxy)ethyl)amino)butyl dihydrogen phosphate; 2-(ethyl(2-((4-((5-(2-((3-fluorophenyl)amino)-2-oxoethyl)-1,3-thiazol-2-yl)amino)-6-methoxyquinazolin-7-yl)oxy)ethyl)amino)ethyl dihydrogen phosphate; (1-(3-((4-((5-(2-((3-fluorophenyl)amino)-2-oxoethyl)-1,3-thiazol-2-yl)amino)quinazolin-7-yl)oxy)propyl)piperidin-4-yl)methyl dihydrogen phosphate; and 2-{4-[({4-[(5-{2-[(3-fluorophenyl)amino]-2-oxoethyl}-1,3-thiazol-2-yl)amino]-6-methoxyquinazolin-7-yl}oxy)methyl]piperidin-1-yl}ethyl dihydrogen phosphate.

Still other non-limiting examples of synthetic VEGF tyrosine kinase inhibitors include the cinnoline derivatives disclosed in U.S. Pat. No. 6,514,971; which is incorporated herein by reference in its entirety. Non-limiting examples of useful cinnoline derivatives are 4-(3-bromoanilino)cinnoline, 6-chloro-4-phenoxycinnoline, 4-anilinocinnolines, 4-phenylthiocinnolines, 4-phenoxycinnolines, 4-(4-methoxyanilino)-6,7-dimethoxycinnoline, and 4-(3-chloroanilino)-6,7-dimethoxycinnoline.

Other VEGF tyrosine kinase inhibitors include antibodies or antibody fragments that bind to the extracellular domains of VEGF receptors. Non-limiting examples of such antibodies and antibody fragments include those disclosed in U.S. Pat. No. 6,448,077 and U.S. Patent Application Publications 2005/0233921, 2005/0244475, and 2006/0014252, which are incorporated herein by reference in their entirety, and their functional equivalents. The term “functional equivalents” of an antibody means polypeptides that have at least 70 percent (or alternatively, at least 80 percent, or at least 90 percent, or at least 95 percent) binding affinity of the antibody toward a target.

In one embodiment, the VEGF receptor tyrosine kinase inhibitor is 3-[(2,4-dimethylpyrrol-5-yl)methylidene]indoline-2-one, known as SU5416, developed by SUGEN, Inc. (South San Francisco, Calif.). This inhibitor has been shown to inactivate effectively the VEGFR-2 receptor. In another embodiment, the VEGF receptor tyrosine kinase inhibitor is a compound of the family of substituted 4-anilinoquinazolines developed by AstraZeneca Pharmaceuticals (Macclesfield, UK), such as the compounds known as ZD4190, ZD6464, ZD6474, and ZD1839. In still another embodiment, the VEGF receptor tyrosine kinase inhibitor is a compound known as ZK222584 or CGP41251, under development by Novartis Pharmaceuticals (Basel, Switzerland).

### 3. Compounds that Reduce a Level of Expression of VEGF

In still another aspect of the present invention, compounds that reduce a level of expression of VEGF comprise those that interfere with the transcription of the VEGF gene and/or translation of a VEGF mRNA. A polynucleotide or oligonucleotide analogue can be used to reduce expression from a selected nucleic acid having a known sequence. As used herein, “reduction” or “reduce” with respect to expression from a nucleic acid refers to a decrease in expression, or to decrease expression, in an amount that can be detected by assessing changes in RNA level, protein level, or phenotype. For example, reduction can refer to at least about 50 percent (or 60 percent, 70 percent, 80 percent, 90 percent), or more than about 95 percent decrease in expression. A reduction in expression also includes substantially complete inhibition of expression, whereby greater than 97 percent (or greater than 99 percent) reduction of expression from a nucleic acid is achieved.

As used herein, the term “expression,” with respect to expression of a gene or expression from a nucleic acid, refers to production of a functional RNA molecule from a DNA molecule as well as production of a functional polypeptide from an mRNA molecule. Expression from a selected nucleic acid can be examined using standard methods known in the art. For example, RNA levels can be determined by Northern hybridization and in situ hybridization using the appropriate nucleic acid hybridization probes, while polypeptide levels can be determine by antibody staining and Western hybridization. Development of organs, differentiated tissues, and other cellular structures that are affected by reduction in expression of selected nucleic acids can be assessed using various methods, including examination of the cells, organs, or tissues or their physiological activity. For example, vasculature can be visualized with FITC (fluorescein isothiocyanate)-dextran injections; cartilage can be visualized using Alcian Blue staining; and muscles can be visualized using fluorescent-phalloidin staining. Alternatively, the expression of tissue-specific genes can be used to assess development of organs, differentiated tissues, and particular cellular structures. For example, expression of VEGF can be examined by studying the proliferation, development, or differentiation of endothelial cell culture.

Expression VEGF from a nucleic acid can be reduced by interfering with (1) any process necessary for RNA transcription, (2) RNA processing, (3) RNA transport across the nuclear membrane, (4) any process necessary for RNA translation, or (5) RNA degradation.

In one aspect, the transcription of the VEGF gene can be affected by hybridizing a small single-stranded nucleotide sequence to the VEGF gene (or in other words, a VEGF antigene oligonucleotide). For example, in one embodiment, such a single-stranded oligonucleotide can be designed to bind to the transcription factor that is responsible for the expression of the VEGF gene, resulting in a lower level of transcription and translation of the VEGF gene. In another embodiment, such a single-stranded oligonucleotide can be designed to bind to the promoter region of the VEGF gene, leading to a reduction or elimination of the transcription of VEGF. In still another embodiment, such a single-stranded oligonucleotide can have the sequence that is complementary to the antisense DNA strand from which VEGF mRNA is transcribed. In yet another embodiment, such a single-stranded oligonucleotide can be an antisense sequence of the coding sequence and at least a non-coding sequence of the VEGF gene. Useful oligonucleotides can have from about 8 to about 120 bases in length, or from about 12 to about 80 bases, or from about 16 to about 60 bases, or from about 20 to about 30 bases. A single-stranded oligonucleotide that binds to the VEGF gene can be designed and synthesized based on the known sequence of the VEGF nucleic acid. See; e.g., U.S. Patent Application Publication 2005/0096257, which is incorporated herein by reference in its entirety, for the sequence of human VEGF nucleic acid.

Expression of VEGF from a nucleic acid such as an RNA molecule also can be reduced by interfering with any process necessary for formation of a functional RNA molecule or proper translation of an mRNA molecule into functional VEGF. Expression from an RNA molecule, for example, can be reduced by interfering with RNA processing, ribosome binding to the ribosome-binding site of mRNAs, interfering with initiation of translation, interfering with the translation process, or interfering with proper termination of translation. A polynucleotide or oligonucleotide or their analogues that hybridize to a region of an mRNA molecule and interferes with its translation has a sequence that is complementary to that region of the mRNA molecule. Such a complementary polynucleotide or oligonucleotide or their analogues (antisense molecules), for example, can bind and sterically inhibit scanning of the mRNA by the ribosomal subunit. As used herein, a polynucleotide or oligonucleotide “analogue” is a chemically modified polynucleotide or oligonucleotide, as the case may be, that has all or portions of the five-carbon sugar-phosphate backbone of the polynucleotide or oligonucleotide replaced with alternate functional groups in such a way that base pairing with the RNA is maintained.

In another aspect, the transcription of the VEGF gene can be affected by an interaction with one of the small organic compounds disclosed in U.S. Patent Application Publication 2005/0282849 or their pharmaceutically acceptable salt, hydrate, solvate, clathrate, racemate, or stereoisomer. This patent application publication is incorporated herein by reference in its entirety. For example, such small organic compounds are represented generally by Formula I.

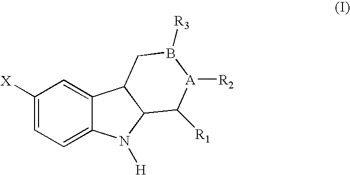

wherein X is hydrogen; a C₁ to C₆ alkyl, optionally substituted with one or more halogens; a hydroxyl group; a halogen; a C₁ to C₅ alkoxy, optionally substituted with a C₆ to C₁₀ aryl group;

### A is C or N;

### B is C or N, with the proviso that at least one of A or B is N, and that when A is N, B is C;

R₁ is a hydroxyl group; a C₁₋₈ alkyl group, optionally substituted with an alkylthio group, a 5 to 10 membered heteroaryl, a C₆₋₁₀ aryl group optionally substituted with at least one independently selected Rₒ group; a C₂₋₈ alkyenyl group; a C₂₋₈ alkynyl group; a 3 to 12 membered heterocycle group, wherein the heterocycle group is optionally substituted with at least one independently selected halogen, oxo, amino, alkylamino, acetamino, thio, or alkylthio group; a 5 to 12 membered heteroaryl group, wherein the heteroaryl group is optionally substituted with at least one independently selected halogen, oxo, amino, alkylamino, acetamino, thio, or alkylthio group; or a C₆ to C₁₀ aryl group, optionally substituted with at least one independently selected Rₒ group;
Rₒ is a halogen; a cyano; a nitro; a sulfonyl, wherein the sulfonyl is optionally substituted with a C₁₋₆ alkyl or a 3 to 10 membered heterocycle; an amino group, wherein the amino group is optionally substituted with a C₁₋₆ alkyl, —C(O)—R_{b}, —C(O)O—R_{b}, a sulfonyl, an alkylsulfonyl, a 3 to 10 membered heterocycle group optionally substituted with a —C(O)O—Rₙ; —C(O)—NH—R_{b}; a 5 to 6 membered heterocycle; a 5 to 6 membered heteroaryl; a C₁₋₆ alkyl group, wherein the alkyl group is optionally substituted with at least one independently selected hydroxyl, halogen, amino, or 3 to 12 membered heterocycle group, wherein the amino group and heterocycle group are optionally substituted with at least one independently selected C₁₋₄ alkyl group, which C₁₋₄ alkyl group is optionally substituted with at least one independently selected C₁₋₄ alkoxy group, amino group, alkylamino group, or 5 to 10 membered heterocycle group; a —C(O)—Rₙ group; or an ORₐ group;
Rₐ is hydrogen; C₂₋₈ alkylene; a —C(O)O—R_{b} group; a —C(O)—NH—R_{b}; a C₁₋₆ alkyl, wherein the alkyl group is optionally substituted with at least one independently selected hydroxyl, halogen, C₁₋₄ alkoxy, amino, alkylamino, acetamide, —C(O)—R_{b}, —C(O)O—R_{b}, C₆₋₁₀ aryl, 3 to 12 membered heterocycle, or 5 to 12 heteroaryl group, further wherein the alkylamino is optionally substituted with a hydroxyl, a C₁₋₄ alkoxy, or a 5 to 12 membered heteroaryl optionally substituted with a C₁₋₄ alkyl, further wherein the acetamide is optionally substituted with a C₁₋₄ alkoxy, sulfonyl, or alkylsulfonyl, further wherein and the heterocycle group is optionally substituted with a C₁₋₄ alkyl optionally substituted with a hydroxyl group, —C(O)—Rₙ, —C(O)O—Rₙ, or an oxo group;
R_{b} is hydroxyl; an amino; an alkylamino, wherein the alkylamino is optionally substituted with a hydroxyl, an amino, an alkylamino, a C₁₋₄ alkoxy, a 3 to 12 membered heterocycle optionally substituted with at least one independently selected C₁₋₆ alkyl, oxo, —C(O)O—Rₙ, or a 5 to 12 membered heteroaryl optionally substituted with a C₁₋₄ alkyl; a C₁₋₄ alkoxy; a C₂₋₈ alkenyl; a C₂₋₈ alkynyl; a C₆₋₁₀ aryl, wherein the aryl is optionally substituted with at least one independently selected halogen or C₁₋₄ alkoxy; a 5 to 12 membered heteroaryl; 3 to 12 membered heterocycle group, wherein the heterocycle is optionally substituted with at least one independently selected acetamide, —C(O)O—Rₙ, 5 to 6 membered heterocycle, or C₁₋₆ alkyl optionally substituted with a hydroxyl, C₁₋₄ alkoxy, amino group, or alkylamino group; or a C₁₋₈ alkyl, wherein the alkyl is optionally substituted with at least one independently selected C₁₋₄ alkoxy, C₆₋₁₀ aryl, amino, or 3 to 12 membered heterocycle group, wherein the amino and heterocycle groups are optionally substituted with at least one independently selected C₁₋₆ alkyl, oxo, or —C(O)O—Rₙ group;
R₂ is a hydrogen; a hydroxyl; a 5 to 10 membered heteroaryl group; a C₁₋₈ alkyl group, wherein the alkyl group is optionally substituted with a hydroxyl, a C₁₋₄ alkoxy, a 3 to 10 membered heterocycle, a 5 to 10 membered heteroaryl, or C₆₋₁₀ aryl group; a —C(O)—R_{c} group; a —C(O)O—R_{d} group; a —C(O)—N(R_{d}) group; a —C(S)—N(R_{d}R_{d}) group; a —C(S)O—Rₑ group; a —S(O₂)—Rₑ group; a —C(NRₑ)—S—Rₑ group; or a —C(S)—S—R_{f} group;
R_{c} is hydrogen; an amino, wherein the amino is optionally substituted with at least one independently selected C₁₋₆ alkyl or C₆₋₁₀ aryl group; a C₆₋₁₀ aryl, wherein the aryl is optionally substituted with at least one independently selected halogen, haloalkyl, hydroxyl, C₁₋₄ alkoxy, or C₁₋₆ alkyl group; —C(O)—Rₙ; a 5 to 6 membered heterocycle, wherein the heterocycle is optionally substituted with a —C(O)—Rₙ group; a 5 to 6 membered heteroaryl; a thiazoleamino group; a C₁₋₈ alkyl group, wherein the alkyl group is optionally substituted with at least one independently selected halogen, a C₁₋₄ alkoxy, a phenyloxy, a C₆₋₁₀ aryl, —C(O) —Rₙ, —O—C(O)—Rₙ, hydroxyl, or amino group, optionally substituted with a —C(O)O—Rₙ group;
R_{d} is independently hydrogen; a C₂₋₈ alkenyl group; a C₂₋₈ alkynyl group; a C₆₋₁₀ aryl group, wherein the aryl is optionally substituted with at least one independently selected halogen, nitro, C₁₋₆ alkyl, —C(O)O—Rₑ, or —ORₑ; or a C₁₋₈ alkyl group, wherein the alkyl group is optionally substituted with at least one independently selected halogen, C₁₋₄ alkyl, C₁₋₄ alkoxy, phenyloxy, C₆₋₁₀ aryl, 5 to 6 membered heteroaryl, —C(O)—Rₙ, —O—C(O)—Rₙ, or hydroxyl group, wherein the C₆₋₁₀ aryl group is optionally substituted with at least one independently selected halogen or haloalkyl group;
Rₑ is a hydrogen; a C₁₋₆ alkyl group, wherein the alkyl group is optionally substituted with at least one independently selected halogen or alkoxy group; or a C₆₋₁₀ aryl group, wherein the aryl group is optionally substituted with at least one independently selected halogen or alkoxy group;
R_{f} is a C₁₋₆ alkyl group, optionally substituted with at least one independently selected halogen, hydroxyl, C₁₋₄ alkoxy, cyano, C₆₋₁₀ aryl, or —C(O)—Rₙ group, wherein the alkoxy group may be optionally substituted with at least one C₁₋₄ alkoxy group and the aryl group may be optionally substituted with at least one independently selected halogen, hydroxyl, C₁₋₄ alkoxy, cyano, or C₁₋₆ alkyl group;

### Rₙ is a hydroxyl, C₁₋₄ alkoxy, amino, or C₁₋₆ alkyl group;

### R₃ is hydrogen or —C(O)—R_{g};

R_{g} is a hydroxyl group; an amino group, wherein the amino is optionally substituted with a C₆₋₁₀ cycloalkyl group or a 5 to 10 membered heteroaryl group; or a 5 to 10 membered heterocycle group, wherein the heterocycle group is optionally substituted with a —C(O)—Rₙ group; and
n is 0, 1, 2, or 3.

In another embodiment, the translation of VEGF mRNA can be interfered by inducing the degradation of VEGF mRNA by a double-stranded RNA (“dsRNA”) that corresponds to the single-stranded mRNA sequence. Following the introduction of the dsRNA into the endothelial cell, the dsRNA is cleaved into single-stranded pieces of RNA. These oligonucleotides bind to and then cleave the VEGF mRNA, resulting in its breakdown.

In still another embodiment, a phosphorothioate antisense DNA oligonucleotide hybridizes to the target site on the VEGF RNA, the RNA-DNA duplex activates the endogenous enzyme ribonuclease H (“RNase H”), which cleaves the mRNA component of the hybrid molecule. A phosphorothioate oligonucleotide has a sulfur group in place of the free oxygen of the phosphodiester bond of the normal oligonucleotide. Such a substitution renders the phosphorothioate more resistant to degradation by intracellular nucleases. Again, such an antisense oligonucleotide can be synthesized from the known sequence of the VEGF DNA.

In yet another aspect, a compound that reduces a level of expression of VEGF can be a VEGF ribozyme. Ribozymes are catalytic RNA (RNA enzyme) that have separate catalytic and substrate-binding domains. Many ribozymes are naturally occurring. In addition, ribozymes can be engineered to specifically cleave various mRNA sequences, such as the VEGF mRNA sequence, at the phosphodiester bond. Methods for preparing ribozymes are disclosed, for example, in U.S. Pat. Nos. 5,037,746; 5,093,246; 5116,742; 5,591,610; 6,025,167; 6,180,399; and 6,696,250; which are incorporated herein by reference in their entirety. In one embodiment, the ribozyme forms a ribozyme-mRNA substrate complex that has the well-known hammerhead or hairpin motif. The frequently used hammerhead ribozymes cleave mRNAs at locations dictated by flanking regions that form complementary base pairs with the target VEGF mRNA. In one aspect of the present invention, ribozymes are delivered to endothelial cells expressing VEGF mRNAs. A useful method of delivery involves using a DNA construct encoding the ribozyme under the control of a strong constitutive pol III or pol II promoter, so that transfected endothelial cells will produce sufficient quantities of the ribozyme to destroy targeted VEGF mRNAs and inhibit their translation. Because ribozymes, unlike antisense molecules, are catalytic, a lower intracellular concentration is required for efficiency.

In another aspect, a composition of the present invention comprises at least a compound that interacts with and inhibits a downstream activity of extracellular VEGF and at least a compound that interacts with at least a VEGF receptor and renders it substantially unavailable for interacting with VEGF.

In still another aspect, a composition of the present invention comprises at least a compound that interacts with at least a VEGF receptor and renders it substantially unavailable for interacting with VEGF and at least a compound that reduces a level of expression of VEGF.

In still another aspect, a composition of the present invention comprises at least a compound that interacts with and inhibits a downstream activity of extracellular VEGF and at least a compound that reduces a level of expression of VEGF.

In a further aspect, a composition of the present invention comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF, wherein said at least two therapeutic agents are selected from the group consisting of polypeptides, oligopeptides, polynucleotides, oligonucleotides, analogues thereof, and combinations thereof.

In still another embodiment, a composition of the present invention comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF, wherein when a compound that reduces a level of expression of VEGF is present, such a compound is selected from the group consisting of polypeptides, oligopeptides, polynucleotides, oligonucleotides, analogues thereof, and combinations thereof.

An advantage of a combination therapy of the present invention is realized in that by substantially simultaneously targeting more than one source of VEGF activity, the dose of each active agent that is effective for reducing the activity of VEGF from each source can be lowered to a non-toxic level. A combination therapy of the present invention also has an advantage of reducing the availability of VEGF more completely, and thus is more effective, than a therapy relying on targeting only one source of VEGF availability.

In yet another aspect, a composition of the present invention further comprises a physiological buffer, such as phosphate buffer or a Tris-HCl buffer (comprising tris(hydroxymethyl)aminomethane and HCI). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5× PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane) ) having pKₐ of 7.52 at 25° C. and pH in the range of about 7-8.2; TAPSO (2-hydroxy-3{tris(hydroxymethyl)methylamino}-1-propanesulfonic acid) ) having pKₐ of 7.61 at 25° C. and pH in the range of about 7-8.2; TAPS ({(2-hydroxy-1,1-bis(hydroxymethyl)ethyl)amino}-1-propanesulfonic acid) ) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid) ) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In one aspect, the pH of the composition is in the range from about 6.5 to about 11. Alternatively, the pH of the composition is in the range from about 6.5 to about 9, or from about 6.5 to about 8. In another aspect, the composition comprises a buffer having a pH in one of said pH ranges.

It may be advantageous to add a stabilizing or bulking agent to a composition of the present invention. Non-limiting examples of such stabilizing or bulking agents are polyhydric alcohols, pharmaceutically acceptable carbohydrates, and combinations thereof. Sugars or sugar alcohols that may be added include glucose, maltose, mannitol, sorbitol, sucrose, lactose, trehalose, and combinations thereof. Other carbohydrates that may be used are polysaccharides, such as dextrin, dextran, glycogen, starches, carboxymethylcellulose, derivatives thereof, and combinations thereof. Concentrations of a carbohydrate added to add bulk to a composition of the present invention can be in a range from about 0.2 percent weight/volume (“% w/v”) to about 20% w/v.

In another aspect, the present invention provides a method for treating or ameliorating a disease condition involving angiogenesis. The method comprises administering to a subject in need of treating or ameliorating the disease condition a therapeutically effective amount of a composition that comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

A composition useful for a method of the present invention can comprise from about 0.0001 weight percent to about 5 weight percent of each of the active compound. Alternatively, a composition can comprise from about 0.001 weight percent to about 3 weight percent of each of the active compound, or from about 0.005 weight percent to about 2 weight percent of each of the active compound, or from about 0.01 weight percent to about 1 weight percent of each of the active compound, or from about 0.005 weight percent to about 0.5 weight percent of each of the active compound.

In still another aspect, such a disease condition involves tumor growth.

In yet another aspect, such a disease condition is selected from the group consisting of DE, DR, AMD, and combinations thereof.

In still another aspect, the present invention provides a method for treating or ameliorating a disease condition involving angiogenesis, the method comprising administering a composition into the vitreous humor of the eye, thereby treating or ameliorating a disease condition involving angiogenesis, wherein the composition comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

In still another aspect, the present invention provides a method for treating or ameliorating a disease condition involving angiogenesis, the method comprising: (a) providing a composition that comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF; and (b) administering said composition into the vitreous humor of the eye, thereby treating or ameliorating a disease condition involving angiogenesis.

In a further aspect, the present invention provides a method for preparing a composition useful for treating or ameliorating a disease condition involving angiogenesis. The method comprises combining at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

In still a further aspect, the method further comprises combining a physiologically acceptable carrier with said at least two therapeutic agents. The concentration of each of the therapeutic agents can be selected from the ranges disclosed above.

Method of injecting a composition of the present invention into the eye for treating or ameliorating a pathological condition involving ocular angiogenesis is now described.

A composition comprising at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF can be injected intravitreally, for example through the pars plana of the ciliary body, using a fine-gauge needle, such as 25-30 gauge. Administration of such a composition can be used to prevent, treat, or ameliorate the potentially blinding complications of an ocular condition, such as DE, DR, AMD, or combinations thereof. A prevention of a disease condition involving angiogenesis may be initiated when an excessive amount of in a tissue or its environment is detected. Typically, an amount from about 25 μl to about 200 μl of a composition of the present invention is administered. The amount of composition comprises each of the active compounds at a concentration effective to treat or ameliorate the pathological condition. Such administration of the composition may be repeated to achieve a substantially full effect upon assessment of the treatment results and recommendation by a skilled medical practitioner.

Tables 1-11 show non-limiting examples of compositions of the present invention, which can be used in the practice of the methods of the present invention disclosed above.

TABLE 1

| Ingredient | Amount per ml | | % composition |
|---|---|---|---|
| Macugen ® | 2 | mg | 0.2 |
| trehalose | 20 | mg | 2 |
| sodium acetate | 2.4 | mg | 0.24 |
| 4-(2-phenyl-1H- | 3 | mg | 0.3 |
| imidazol-1-yl)-N- | | | |
| pyridin-4-ylpyrimidin- | | | |
| 2-amine (a tyrosine | | | |
| kinase inhibitor) | | | |
| normal saline | QS to 1 | ml | 97.26 |

TABLE 2

| Ingredient | Amount per ml | | % composition |
|---|---|---|---|
| Macugen ® | 2 | mg | 0.2 |
| trehalose | 20 | mg | 2 |
| sodium acetate | 2.4 | mg | 0.24 |
| 4-(2-phenyl-1H- | 3 | mg | 0.3 |
| imidazol-1-yl)-N- | | | |
| pyrimidin-4- | | | |
| ylpyrimidin-2-amine | | | |
| (a tyrosine kinase | | | |
| inhibitor) | | | |
| normal saline | QS to 1 | ml | 97.26 |

TABLE 3

| Ingredient | Amount per ml | | % composition |
|---|---|---|---|
| Macugen ® | 4 | mg | 0.4 |
| trehalose | 20 | mg | 2 |
| sodium acetate | 2.4 | mg | 0.24 |
| 4-(2-phenyl-1H- | 3 | mg | 0.3 |
| imidazol-1-yl)-N- | | | |
| pyrazin-2-ylpyrimidin- | | | |
| 2-amine (a tyrosine | | | |
| kinase inhibitor) | | | |
| phosphate buffer (pH | QS to 1 | ml | 97.06 |
| 7.4) | | | |

TABLE 4

| Ingredient | Amount per ml | | % composition |
|---|---|---|---|
| Lucentis ™ | 2 | mg | 0.2 |
| trehalose | 20 | mg | 2 |
| sodium acetate | 2.4 | mg | 0.24 |
| N-isoxazol-3-yl-4-(2- | 3 | mg | 0.3 |
| phenyl-1H-imidazol- | | | |
| 1-yl)pyrimidin-2- | | | |
| amine (a tyrosine | | | |
| kinase inhibitor) | | | |
| dsRNA having | 2 | mg | 0.2 |
| sequence | | | |
| corresponding to | | | |
| single-stranded | | | |
| VEGF mRNA | | | |
| phosphate buffer (pH | QS to 1 | ml | 97.06 |
| 7.4) | | | |

TABLE 5

| Ingredient | Amount per ml | | % composition |
|---|---|---|---|
| Macugen ® | 10 | mg | 1 |
| trehalose | 20 | mg | 2 |
| sodium acetate | 2.4 | mg | 0.24 |
| VEGF ribozyme | 3 | mg | 0.3 |
| normal saline | QS to 1 | ml | 96.46 |

TABLE 6

| Ingredient | Amount per ml | | % composition |
|---|---|---|---|
| Macugen ® | 2 | mg | 0.2 |
| trehalose | 20 | mg | 2 |
| sodium acetate | 2.4 | mg | 0.24 |
| N-(3-methylisoxazol- | 3 | mg | 0.3 |
| 5-yl)-4-(2-phenyl-1H- | | | |
| imidazol-1- | | | |
| yl)pyrimidin-2-amine | | | |
| (a tyrosine kinase | | | |
| inhibitor) | | | |
| VEGF ribozyme | 3 | mg | 0.3 |
| normal saline | QS to 1 | ml | 96.96 |

TABLE 7

| Ingredient | Amount per ml | | % composition |
|---|---|---|---|
| Macugen ® | 2 | mg | 0.2 |
| trehalose | 20 | mg | 2 |
| sodium citrate | 2.4 | mg | 0.24 |
| Lucentis ™ | 3 | mg | 0.3 |
| N-(4-methyl-1,3- | 5 | mg | 0.5 |
| thiazol-2-yl)-4-(2- | | | |
| phenyl-1H-imidazol- | | | |
| 1-yl)pyrimidin-2- | | | |
| amine (a tyrosine | | | |
| kinase inhibitor) | | | |
| VEGF ribozyme | 3 | mg | 0.3 |
| N-(2,6- | 3 | mg | 0.3 |
| dimethylpyridin-4-yl)- | | | |
| 4-(2-phenyl-1H- | | | |
| imidazol-1- | | | |
| yl)pyrimidin-2-amine | | | |
| (a tyrosine kinase | | | |
| inhibitor) | | | |
| normal saline | QS to 1 | ml | 96.16 |

TABLE 8

| Ingredient | Amount per ml | | % composition |
|---|---|---|---|
| Lucentis ™ | 2 | mg | 0.2 |
| mannitol | 20 | mg | 2 |
| sodium acetate | 2.4 | mg | 0.24 |
| N-{3-methoxy-5- | 3 | mg | 0.3 |
| (trifluoromethyl)phenyl}- | | | |
| 4-(2-phenyl-1H- | | | |
| imidazol-1-yl)pyrimidin- | | | |
| 2-amine (a tyrosine | | | |
| kinase inhibitor) | | | |
| VEGF ribozyme | 5 | mg | 0.5 |
| normal saline | QS to 1 | ml | 96.76 |

TABLE 9

| Ingredient | Amount per ml | | % composition |
|---|---|---|---|
| Macugen ® | 2 | mg | 0.2 |
| trehalose | 20 | mg | 2 |
| sodium acetate | 2.4 | mg | 0.24 |
| 4-(2-phenyl-1H- | 3 | mg | 0.3 |
| imidazol-1-yl)-N-{3- | | | |
| (trifluoromethyl)phenyl} | | | |
| pyrimidin-2-amine (a | | | |
| tyrosine kinase | | | |
| inhibitor) | | | |
| normal saline | QS to 1 | ml | 97.26 |

TABLE 10

| Ingredient | Amount per ml | | % composition |
|---|---|---|---|
| Lucentis ™ | 2 | mg | 0.2 |
| trehalose | 20 | mg | 2 |
| sodium acetate | 2.4 | mg | 0.24 |
| (3-methyl-5-{(4-(2- | 3 | mg | 0.3 |
| phenyl-1H-imidazol-1- | | | |
| yl)pyrimidin-2- | | | |
| yl)amino}phenyl) | | | |
| methanol (a tyrosine | | | |
| kinase inhibitor) | | | |
| normal saline | QS to 1 | ml | 97.26 |

TABLE 11

| Ingredient | Amount per ml | | % composition |
|---|---|---|---|
| Lucentis ™ | 2 | mg | 0.2 |
| polypeptide antibody | 3 | mg | 0.3 |
| against VEGFR-2 | | | |
| mannitol | 20 | mg | 2 |
| sodium acetate | 2.4 | mg | 0.24 |
| (3-methyl-5-{(4-(2- | 3 | mg | 0.3 |
| phenyl-1H-imidazol- | | | |
| 1-yl)pyrimidin-2- | | | |
| yl)amino}phenyl) | | | |
| methanol (a tyrosine | | | |
| kinase inhibitor) | | | |
| normal saline | QS to 1 | ml | 96.96 |

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising at least two therapeutic agents that target two or more sources of VEGF activity in a disease condition involving angiogenesis.

2. The composition of claim 1, wherein said composition comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

3. The composition of claim 2, wherein said disease condition is selected from the group consisting of diabetic edema (“DE”), diabetic retinopathy (“DR”), age-related macular degeneration (“AMD”), and combinations thereof.

4. The composition of claim 2, wherein said compounds that interact with and inhibit a downstream activity of extracellular VEGF are selected from the group consisting of VEGF antagonist aptamers, anti-VEGF antibodies, anti-VEGF antibody fragments, and mixtures thereof.

5. The composition of claim 2, wherein the compounds that interact with at least a VEGF receptor and render it substantially unavailable for interacting with VEGF are selected from the group consisting of VEGF tyrosine kinase inhibitors, antibody fragments that bind to an extracellular domain of a VEGF receptor, and mixtures thereof.

6. The composition of claim 5, wherein the VEGF tyrosine kinase inhibitors are selected from compounds comprising pyrimidine or substituted pyrimidine linked to imidazole or substituted imidazole.

7. The composition of claim 5, wherein VEGF tyrosine kinase inhibitors are selected from quinazoline derivatives, cinnoline derivatives, and mixtures thereof.

8. The composition of claim 2, wherein the compounds that reduce a level of expression of VEGF are selected from the group consisting of VEGF antigene oligonucleotides, VEGF antigene polynucleotides, double-stranded RNAs that have a sequence corresponding to VEGF mRNA sequence, antisense DNAs that are capable of hybridize to VEGF mRNA, VEGF ribozymes, and mixtures thereof.

9. The composition of claim 2, wherein each of said therapeutic agents is present in the composition in an amount from about 0.0001 weight percent to about 5 weight percent.

10. The composition of claim 2, wherein each of said therapeutic agents is present in the composition in an amount from about 0.001 weight percent to about 3 weight percent.

11. The composition of claim 1, wherein said composition comprises at least two therapeutic agents selected from the group consisting of compounds that interact with and inhibit a downstream activity of extracellular VEGF and compounds that interact with at least a VEGF receptor and render it substantially unavailable for interacting with VEGF.

12. The composition of claim 1, wherein said composition comprises at least two therapeutic agents selected from the group consisting of compounds that interact with and inhibit a downstream activity of extracellular VEGF and compounds that reduce a level of expression of VEGF.

13. The composition of claim 1, wherein said composition comprises at least two therapeutic agents selected from the group consisting of compounds that interact with at least a VEGF receptor and render it substantially unavailable for interacting with VEGF and compounds that reduce a level of expression of VEGF.

14. The composition of claim 13, wherein at least two therapeutic agents are selected from the group consisting of polypeptides, oligopeptides, polynucleotides, oligonucleotides, and combinations thereof.

15. A composition comprising at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF; wherein said compounds that interact with and inhibit a downstream activity of extracellular VEGF are selected from the group consisting of VEGF antagonist aptamers, anti-VEGF antibodies, anti-VEGF antibody fragments, and mixtures thereof; said compounds that interact with at least a VEGF receptor and render it substantially unavailable for interacting with VEGF are selected from the group consisting of VEGF tyrosine kinase inhibitors, antibody fragments that bind to an extracellular domain of a VEGF receptor, and mixtures thereof; said compounds that reduce a level of expression of VEGF are selected from the group consisting of VEGF antigene oligonucleotides, VEGF antigene polynucleotides, double-stranded RNAs that have a sequence corresponding to VEGF mRNA sequence, antisense DNAs that are capable of hybridize to VEGF mRNA, VEGF ribozymes, and mixtures thereof; and each of said therapeutic agents is present in the composition in an amount from about 0.0001 weight percent to about 5 weight percent.

16. A composition comprising: compounds that interact with and inhibit a downstream activity of extracellular VEGF, compounds that interact with at least a VEGF receptor and render it substantially unavailable for interacting with VEGF, and compounds that reduce a level of expression of VEGF.

17. The composition of claim 16, wherein the compounds that reduce a level of expression of VEGF are selected from the group consisting of VEGF antigene oligonucleotides, VEGF antigene polynucleotides, double-stranded RNAs that have a sequence corresponding to VEGF mRNA sequence, antisense DNAs that are capable of hybridize to VEGF mRNA, VEGF ribozymes, and mixtures thereof.

18. A method for producing a composition for use in treating, preventing, or ameliorating a disease condition involving angiogenesis, the method comprising combining at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

19. The method of claim 18, further comprising combining a physiologically acceptable carrier with said at least two therapeutic agents.

20. Use of at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF to produce a composition for treating, preventing, or ameliorating a disease condition involving angiogenesis in a subject in need therefor.

21. A method for treating, preventing, or ameliorating a disease condition involving angiogenesis, the method comprising administering a composition into the vitreous humor of the eye, thereby treating, preventing, or ameliorating said disease condition, wherein the composition comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

22. The method of claim 21, wherein said disease condition is selected from the group consisting of diabetic edema (“DE”), diabetic retinopathy (“DR”), age-related macular degeneration (“AMD”), and combinations thereof.

23. The method of claim 22, wherein said compounds that interact with and inhibit a downstream activity of extracellular VEGF are selected from the group consisting of VEGF antagonist aptamers, anti-VEGF antibodies, anti-VEGF antibody fragments, and mixtures thereof.

24. The method of claim 22, wherein the compounds that interact with at least a VEGF receptor and render it substantially unavailable for interacting with VEGF are selected from the group consisting of VEGF tyrosine kinase inhibitors, antibody fragments that bind to an extracellular domain of a VEGF receptor, and mixtures thereof.

25. The method of claim 24, wherein the VEGF tyrosine kinase inhibitors are selected from compounds comprising pyrimidine or substituted pyrimidine linked to imidazole or substituted imidazole.

26. The method of claim 24, wherein VEGF tyrosine kinase inhibitors are selected from quinazoline derivatives, cinnoline derivatives, and mixtures thereof.

27. The method of claim 22, wherein the compounds that reduce a level of expression of VEGF are selected from the group consisting of VEGF antigene oligonucleotides, VEGF antigene polynucleotides, double-stranded RNAs that have a sequence corresponding to VEGF mRNA sequence, antisense DNAs that are capable of hybridize to VEGF mRNA, VEGF ribozymes, and mixtures thereof.

28. The method of claim 22, wherein each of said therapeutic agents is present in the composition in an amount from about 0.0001 weight percent to about 5 weight percent.

29. The method of claim 22, wherein each of said therapeutic agents is present in the composition in an amount from about 0.001 weight percent to about 3 weight percent.

30. The method of claim 20, further comprising providing the composition before the step of administering.

31. The method of claim 30, wherein the step of providing comprises combining said at least two therapeutic agents.

32. The method of claim 30, wherein the step of providing comprises combining said at least two therapeutic agents with a physiologically acceptable carrier.

33. A method for treating, preventing, or ameliorating a disease condition involving angiogenesis, the method comprising administering a composition into a subject, thereby treating, preventing, or ameliorating said disease condition in said subject, wherein the composition comprises at least two therapeutic agents selected from the group consisting of compounds that interact with and inhibit a downstream activity of extracellular VEGF, compounds that interact with at least a VEGF receptor and render it substantially unavailable for interacting with VEGF, and compounds that reduce a level of expression of VEGF.

34. The method of claim 33, wherein the compounds that reduce a level of expression of VEGF are selected from the group consisting of VEGF antigene oligonucleotides, VEGF antigene polynucleotides, double-stranded RNAs that have a sequence corresponding to VEGF mRNA sequence, antisense DNAs that are capable of hybridize to VEGF mRNA, VEGF ribozymes, and mixtures thereof.

35. A method for treating, preventing, or ameliorating a disease condition involving angiogenesis, the method comprising administering a composition into a subject, thereby treating, preventing, or ameliorating said disease condition in said subject, wherein the composition comprises: compounds that interact with and inhibit a downstream activity of extracellular VEGF, compounds that interact with at least a VEGF receptor and render it substantially unavailable for interacting with VEGF, and compounds that reduce a level of expression of VEGF.

36. The method of claim 35, wherein the compounds that reduce a level of expression of VEGF are selected from the group consisting of VEGF antigene oligonucleotides, VEGF antigene polynucleotides, double-stranded RNAs that have a sequence corresponding to VEGF mRNA sequence, antisense DNAs that are capable of hybridize to VEGF mRNA, VEGF ribozymes, and mixtures thereof.
